(19) United States
(12) Patent Application Publication
(10) Pub. No.: US 2008/0312616 A1
(43) Pub. Date: Dec. 18, 2008

(54) ABSORBENT UNDER-BREAST PAD

(76) Inventor: Theresa Nixon, Whittier, CA (US)

Correspondence Address: AVERILL & VARN, 8244 PAINTER AVE., WHITTIER, CA, 90602, US

(21) Appl. No.: 11/764,099

(22) Filed: Jun. 15, 2007

(51) Int. Cl. A61F 5/44; A61F 13/15

(52) U.S. Cl. 604/355; 604/387

(57) ABSTRACT

A moisture absorbent under-breast pad provides absorption of moisture between the breast and rib cage. The pad is made from 100 percent cotton cloth and is held in place by the weight of the breasts without adhesives or straps. The pad is comfortable to wear and provides relief from yeast infections, rashes, perspiration odor, and itching.

### BACKGROUND OF THE INVENTION

The present invention relates to moisture absorbing pads and in particular to an under-breast moisture absorbing pad.

Some women with large breast sizes have reoccurring problems resulting from moisture trapped between their breasts and rib cages. These problems include yeast infections, rashes, itching, etc. which result from an accumulation of perspiration and moisture. Ointments, salves, and the like have been used to treat these problems, but produce an uncomfortable sensation, and may discolor clothing.

### BRIEF SUMMARY OF THE INVENTION

The present invention addresses the above and other needs by providing a moisture absorbent under-breast pad which provides absorption of moisture between the breast and rib cage. The pad is made from 100 percent cotton cloth and is held in place by the weight of the breasts without adhesives or straps. The pad is comfortable to wear and provides relief from yeast infections, rashes, and itching.

In accordance with one aspect of the invention, there is provided a moisture absorbent under-breast pad adapted to be held in place between a woman's breasts and rib cage by a weight of the breasts alone for absorbing moisture and prevent skin to skin contact. The moisture absorbent under-breast pad includes a substantially flat layer of soft and absorbent cloth consisting substantially of cotton and having a width of between approximately 15 inches and approximately 18 inches, a straight top edge, and curved bottom edge including two “cup” sections resembling the outline of the woman's breasts. The “cup” sections may include one or more additional layers of fabric to improve comfort.

In accordance with another aspect of the invention, there is provided a method for controlling moisture between breasts and the rib cage. The method includes lifting a woman's breasts, placing an adhesiveless and strapless moisture absorbent under-breast pad between the woman's breasts and rib cage for absorbing moisture and preventing skin to skin contact and releasing the breasts to fall against the adhesiveless and strapless moisture absorbent under-breast pad to hold the adhesiveless and strapless moisture absorbent under-breast pad in place between the breasts and rib cage. The moisture absorbent under-breast pad includes a substantially flat layer of soft and absorbent cloth having approximately a width of the woman's breasts, a straight top edge, and a curved bottom edge resembling the shape of the woman's breasts.

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1A is a front view of a moisture absorbent under-breast pad according to the present invention.

FIG. 1B is a side view of the moisture absorbent under-breast pad according to the present invention.

FIG. 2 is an embodiment of the moisture absorbent under-breast pad according to the present invention with a curved top to fit under the breasts.

FIG. 3 depicts the moisture absorbent under-breast pad residing under a woman's breasts between the breasts and rib cage.

FIG. 4 describes a method for controlling moisture under a woman's breasts according to the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

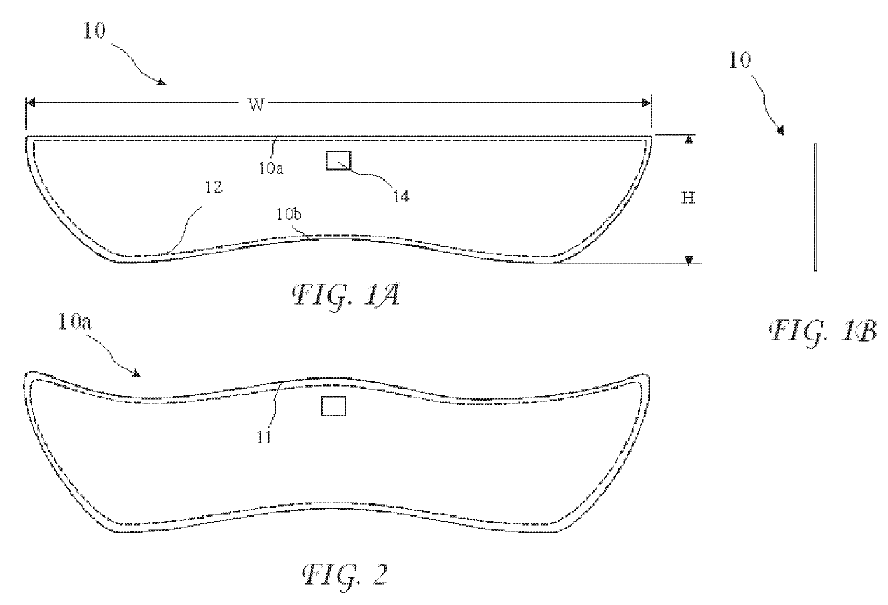

A front view of a moisture absorbent under-breast pad 10 according to the present invention is shown in FIG. 1A and a side view of the moisture absorbent under-breast pad 10 is shown in FIG. 1B. The moisture absorbent under-breast pad 10 is substantially flat with a substantially straight top 10a and a curved bottom 10b somewhat following an outline of a woman's breasts. The top 10a is substantially straight in that it is straight within the normal stretching an cutting of cloth and appears straight. The moisture absorbent under-breast pad 10 is preferably made in three sizes to comfortably fit different women. The moisture absorbent under-breast pad 10 has a width W which is between approximately 15 inches and approximately 18 inches and a height H which is between approximately 3.5 inches to approximately five inches. A seam 12 is sown around the perimeter of the moisture absorbent under-breast pad 10 and a pull 14 resides approximately centered and near the top of the moisture absorbent under-breast pad 10.

The moisture absorbent under-breast pad 10 is made from a soft comfortable and moisture absorbent material which is machine washable. A preferred material is mostly cotton or 100 percent cotton and is preferably organic. An example of a suitable cotton material is between 34 and 36 gauge Jersey knit cotton.

An embodiment of the moisture absorbent under-breast pad 10a according to the present invention with a curved top 11 to fit under the breasts is shown in FIG. 2. The moisture absorbent under-breast pad 10a is otherwise similar to the moisture absorbent under-breast pad 10.

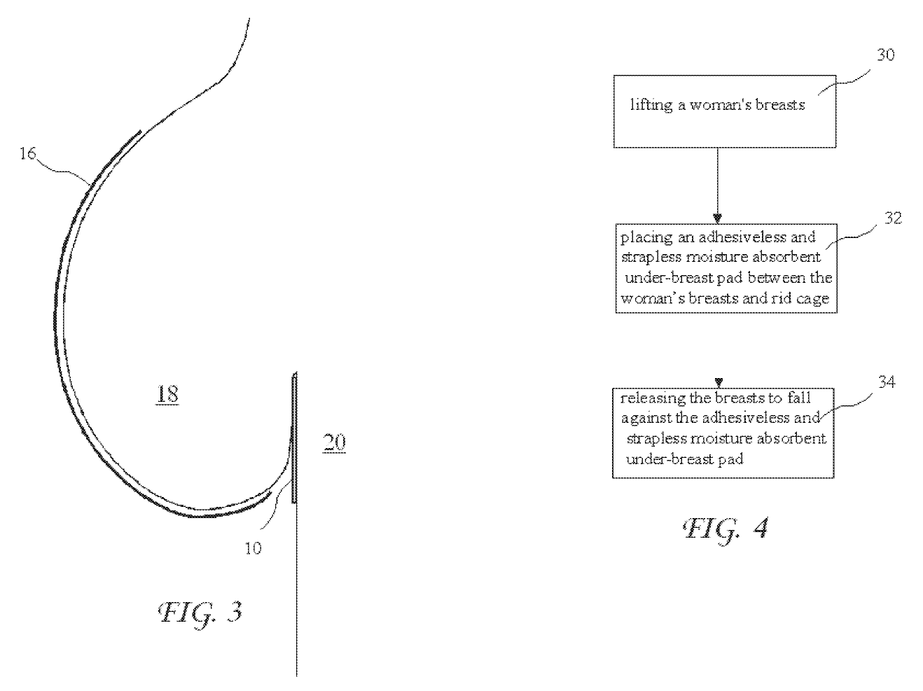

The moisture absorbent under-breast pad 10 is shown residing under a woman's breast 18 between the breast 18 and rib cage 20 and behind a bra 16 in FIG. 3. Thus positioned, the moisture absorbent under-breast pad 10 is held in place without adhesives or straps. The moisture absorbent under-breast pad 10 thus prevents skin to skin contact and absorbs moisture which otherwise would cause discomfort.

A method for controlling moisture under a woman's breasts according to the present invention is described in FIG. 4. The woman's breasts are lifted at step 30. An adhesiveless and strapless moisture absorbent under-breast pad is placed between the woman's breasts and rib cage at step 32. The breasts are released to fall against the adhesiveless and strapless moisture absorbent under-breast pad to hold the adhesiveless and strapless moisture absorbent under-breast pad in place at step 34.

Although the moisture absorbent under-breast pad 10 is preferably strapless, in some instances where a strap is required, a strap may be attached.

While the invention herein disclosed has been described by means of specific embodiments and applications thereof, numerous modifications and variations could be made thereto by those skilled in the art without departing from the scope of the invention set forth in the claims.

I claim:

1. A moisture control comprising:
a moisture absorbent under-breast pad residing between a woman's breasts and rib cage for absorbing moisture and preventing skin to skin contact, the moisture absorbent under-breast pad comprising:
a substantially flat layer of soft and absorbent cloth having approximately a width of the woman's breasts;
a top edge; and
curved bottom edge resembling the shape of the woman's breasts.

2. The moisture control of claim 1, wherein the top edge is substantially straight.

3. The moisture control of claim 1, wherein the top edge is curved to fit under the breasts.

4. The moisture control of claim 1, wherein the moisture absorbent under-breast pad is made of cotton.

5. The moisture control of claim 4, wherein the moisture absorbent under-breast pad is made of 100% cotton.

6. The moisture control of claim 5, wherein the moisture absorbent under-breast pad is made of between 34 and 36 gauge cotton.

7. The moisture control of claim 5, wherein the moisture absorbent under-breast pad is a Jersey knit cotton.

8. The moisture control of claim 1, wherein the moisture absorbent under-breast pad is between approximately 15 inches and approximately 18 inches wide.

9. The moisture control of claim 8, wherein the moisture absorbent under-breast pad is between approximately 3.5 inches and approximately five inches high.

10. The moisture control of claim 1, wherein the moisture absorbent under-breast pad includes a pull approximately centered and near a top edge of the moisture absorbent under-breast pad.

11. The moisture control of claim 1, wherein the moisture absorbent under-breast pad is adhesiveless and strapless and adapted to be held in place between a woman's breasts and rib cage by a weight of the breasts alone.

12. The moisture control of claim 1, wherein the moisture absorbent under-breast pad is organic cotton.

13. A moisture control comprising:
a moisture absorbent under-breast pad adapted to be held in place between a woman's breasts and rib cage by a weight of the breasts alone, the moisture absorbent under-breast pad for absorbing moisture and preventing skin to skin contact, the moisture absorbent under-breast pad comprising:
a substantially flat layer of soft and absorbent cloth consisting substantially of cotton and having a width of between approximately 15 inches and approximately 18 inches;
a straight top edge; and
curved bottom edge include cup sections resembling the outline of the woman's breasts.

14. The moisture control of claim 13, wherein the cup sections include one or more additional layers of fabric.

15. A method for controlling moisture between breasts and a rib cage, the method comprising:
lifting a woman's breasts;
placing an adhesiveless and strapless moisture absorbent under-breast pad between the woman's breasts and rib cage for absorbing moisture and preventing skin to skin contact, the moisture absorbent under-breast pad comprising:
a substantially flat layer of soft and absorbent cloth having approximately a width of the woman's breasts;
a straight top edge; and
curved bottom edge resembling the shape of the woman's breasts; and
releasing the breasts to fall against the adhesiveless and strapless moisture absorbent under-breast pad to hold the adhesiveless and strapless moisture absorbent under-breast pad in place between the breasts and rib cage.
